(19) United States
(12) Patent Application Publication
(10) Pub. No.: US 2012/0159669 A1
(43) Pub. Date: Jun. 21, 2012

(54) METHOD FOR ALTERING DEPTH OF FLOWER COLOR FOR THE LIGHTER OR DEEPER

(75) Inventors: Shigeru Iida, Shizuoka-shi (JP); Yasumasa Morita, Tsukuba-shi (JP); Atsushi Hoshino, Okazaki-shi (JP); Yoshikazu Tanaka, Mishima-gun (JP); Masako Mizutani, Mishima-gun (JP)

(73) Assignee: Suntory Holdings Limited, Osaka-shi (JP)

(21) Appl. No.: 13/378,895

(22) PCT Filed: Jun. 1, 2010
(86) PCT No.: PCT/JP2010/059281

§ 371 Date: Mar. 2, 2012

(30) Foreign Application Priority Data
Jun. 30, 2009 (JP) 2009-155853

(51) Int. Cl. A01H 3/04; C12N 15/61; A01H 5/00

(52) U.S. Cl. 800/282; 800/323.1; 536/23.2; 800/323

(57) ABSTRACT

Disclosed is a novel method for altering the depth of flower color for the lighter or deeper. Specifically disclosed is a method for altering the depth of flower color of a plant for the lighter or deeper by varying the anthocyanin content in petals of the plant, which comprises a step of regulating the expression of type IV chalcone isomerase in the plant.

### FIELD OF THE INVENTION

The present invention relates to a method for altering the depth of flower color of a plant for the lighter or deeper by varying the anthocyanin content in petals of the plant, which comprises a step of regulating the expression of type IV chalcone isomerase in the plant, more specifically to a method for altering the depth of flower color of a plant for the lighter by reducing the anthocyanin content in petals of the plant, which comprises a step of suppressing the expression of type IV chalcone isomerase in the plant, and to a novel gene useful in these methods.

### BACKGROUND OF THE INVENTION

Flower color is an important character of petals, and thus varying flower color is useful in the flower industry. As methods for altering the traits of flowers including flower color, there are (i) crossing, (ii) mutation, (iii) gene recombination technology and the like. Methods for altering the depth of flower color of a plant for the lighter or whiter by gene recombination technology have been realized by suppressing the expression of some of the genes of the enzymes involved in the biosynthesis of anthocyanins which are flower pigments in petunia, torenia, rose, chrysanthemum, carnation and the like.

Chalcone isomerase is one of the enzymes involved in the biosynthesis of anthocyanins, and catalyzes a reaction of isomerizing chalcone to flavonone. A representative chalcone is 2′,4,4′,6′-tetrahydrochalcone, which is isomerized to naringenin by chalcone isomerase. Chalcone isomerase that catalyzes this reaction is referred to as type I chalcone isomerase (hereinafter, see Nonpatent document 1). In addition to this reaction, leguminous plants have a chalcone isomerase that catalyzes a reaction of isomerizing 6′-deoxychalcone to 5-deoxyflavanone. This chalcone isomerase is referred to as type II chalcone isomerase. Amino acid identity between these chalcone isomerase is 55-90% in the case of Lotus japonicum.

In addition to type I and type II chalcone isomerases, there are proteins that exhibit homology with the amino acid sequence of chalcone isomerase. According to molecular phylogeny based on the amino acid sequence, there are groups that are classified as type III and type IV. Both of the groups of type III and type IV chalcone isomerases have no catalytic center of chalcone isomerase type I, and thus are considered to have no activity as the chalcone isomerase (see Nonpatent document 1). As the type IV chalcone isomerase, a protein encoded by AT5G05270 of Arabidopsis is known to have a weak identity, but its function is unknown. It is also unknown whether or not type IV chalcone isomerase is involved in the synthesis of anthocyanins, and there are no reports so far that the gene of type IV chalcone isomerase was used to alter flower color. Furthermore, a plurality of enzymes involved in the synthesis of anthocyanins have been postulated to be in a supermolecular structure, and in such interaction type IV chalcone isomerase is not known to involved.

Morning glory (Ipomoea nil) is one of the most popular garden plants in Japan, and has undergone breeding since the Edo period of Japan. In the process, a variety of mutants with different flower colors and shapes were obtained. The mutagen of the mutable Ipomoea nil among these mutants is known to be a DNA-type transposon related to Tpn 1. For example, when Tpn 1 is inserted into a structural gene such as the gene of chalcone synthase and chalcone isomerase (isomerizing enzyme), the synthesis of anthocyanins is inhibited thereby rendering the flower color whiter. If Tpn 1 is eliminated and the function of the structural gene is restored, anthocyanin synthesis may be resumed resulting in pigmentation. On the other hand, as the gene locus involved in the depth of flower color, Dilute, Intense, Light, Tinged, Fainted etc. are known. However, these genes have not been identified yet and have not been industrially used.

Ipomoea nil is also known to produce anthocyanins of complex structure. The most complex anthocyanin is called heavenly blue anthocyanin, which has six units of glucose and three units of caffeic acid bound to peonidin. When the B ring of heavenly blue anthocyanin lacks a 3′ methoxy group, it is called wedding bell anthocyanin. Though the biosynthetic pathways of these anthocyanins have not been completely elucidated, precursor anthocyanins thereof, i.e. anthocyanins having smaller numbers of bound glucose and caffeic acid units, are contained in petals of Ipomoea nil. The transcription of genes responsible for anthocyanins and flower color is known to be regulated by transcription control factors having the R2R3-Myb domain, bHLH domain or WD40 repeat, and the loci and the genes corresponding to them have been identified. The loci c-1 and ca encode a transcription control factor having the R2R3-Myb domain and a transcription control factor having the WD40 repeat, respectively.

The type and content of anthocyanins in an Ipomoea nil var. Chidori that blooms red flowers, and Kusumi Chidori, a mutable mutant having a lighter depth of flower color, obtained from Chidori by natural mutation, were analyzed by high performance liquid chromatography (HPLC) according to the method described in the following Nonpatent document 2. Prior to the HPLC analysis, anthocyanin was extracted from the enlarged part of the bloomed petal with 95% MEOH/5% acetic acid. Compared to var. Chidori, the content of anthocyanin was reduced in the mutant, but the types of anthocyanins contained in the petal were not different between Chidori and the mutant including wedding bell anthocyanin. This suggests that the lighter depth of flower color in the mutant of an Ipomoea nil var. Chidori may not be due to the mutation of enzymes involved in the biosynthesis of anthocyanins.

### CITING REFERENCES

### Nonpatent Documents

Nonpatent document 1: Plant Physiology, April 2005, Vol. 137, pp. 1375-1386

Nonpatent document 2: Phytochemistry, February 1992, Vol. 31, pp. 659-663

### SUMMARY OF THE INVENTION

### Problems to be Solved by the Invention

The present invention intends to provide a novel method for altering the depth of flower color for the lighter or deeper.

### Means to Solve the Problems

The present inventors have assumed that the genetic difference between the Chidori strain that blooms a red-colored flower and the Kusumi Chidori strain that blooms a pink-colored flower may be caused by the transposon inserted into the causative gene, and thus have amplified a region adjacent to the transposon using the transposon display method to obtain a DNA fragment that is specifically increased in the Kusumi Chidori strain. Since such a DNA fragment did not encode protein, a genomic region in the vicinity was isolated from the red Chidori strain using the inverse PCR method. The genomic region obtained was found to exhibit homology with a chalcone isomerase that is classified into type IV isolated from soy beans. Based on such findings, it was assumed that the depth of flower color may be altered for the lighter by reducing the anthocyanin content in petals of the plant, and after repeated experiments, this was confirmed and the present invention was completed.

Thus, the present invention is as follows:

[1] A method for altering the depth of flower color for the lighter or deeper by varying the anthocyanin content in petals of a plant, which comprises a step of regulating the expression of type IV chalcone isomerase in the plant.

[2] A method for altering the depth of flower color for the lighter by reducing the anthocyanin content in petals of a plant, which comprises a step of suppressing the expression of type IV chalcone isomerase in the plant.

[3] The method according to the above [2] for altering the depth of flower color of a petunia plant for the lighter by reducing the anthocyanin content in petals of the petunia, which comprises a step of suppressing the expression of a gene comprising any of the following (a) to (d):
(a) a DNA comprising the nucleotide sequence set forth in SEQ ID NO: 25;
(b) a DNA that comprises a nucleotide sequence in which one or several nucleotides have been deleted, substituted or added in the nucleotide sequence set forth in SEQ ID NO: 25 and that has a function equivalent to that of a DNA comprising the nucleotide sequence set forth in SEQ ID NO: 25 in terms of the effect of reducing the anthocyanin content;
(c) a DNA that hybridizes to a complementary strand of a DNA comprising the nucleotide sequence set forth in SEQ ID NO: 25 under a stringent condition and that has a function equivalent to that of a DNA comprising the nucleotide sequence set forth in SEQ ID NO: 25 in terms of the effect of reducing the anthocyanin content; and
(d) a DNA that has a sequence identity of at least 90% with a DNA comprising the nucleotide sequence set forth in SEQ ID NO: 25 and that has a function equivalent to that of a DNA comprising the nucleotide sequence set forth in SEQ ID NO: 25 in terms of the effect of reducing the anthocyanin content.

[4] The method according to the above [3] wherein the expression of the above gene is suppressed by the RNAi method.

[5] A petunia obtained by the method described in the above [3] or [4], a progeny thereof having a property equivalent to said petunia in terms of the effect of reducing the anthocyanin content, or a tissue thereof.

[6] A gene comprising any of the following (a) to (d):
(a) a DNA comprising the nucleotide sequence set forth in SEQ ID NO: 25;
(b) a DNA that comprises a nucleotide sequence in which one or several nucleotides have been deleted, substituted or added in the nucleotide sequence set forth in SEQ ID NO: 25 and that has a function equivalent to that of a DNA comprising the nucleotide sequence set forth in SEQ ID NO: 25 in terms of the effect of reducing the anthocyanin content;
(c) a DNA that hybridizes to a complementary strand of a DNA comprising the nucleotide sequence set forth in SEQ ID NO: 25 under a stringent condition and that has a function equivalent to that of a DNA comprising the nucleotide sequence set forth in SEQ ID NO: 25 in terms of the effect of reducing the anthocyanin content; and
(d) a DNA that has a sequence identity of at least 90% with a DNA comprising the nucleotide sequence set forth in SEQ ID NO: 25 and that has a function equivalent to that of a DNA comprising the nucleotide sequence set forth in SEQ ID NO: 25 in terms of the effect of reducing the anthocyanin content.

[7] The method according to the above [2] for altering the depth of flower color of the torenia plant for the lighter by reducing the anthocyanin content in petals of the torenia, which comprises a step of suppressing the expression of a gene comprising any of the following (a) to (d):
(a) a DNA comprising the nucleotide sequence set forth in SEQ ID NO: 29 or 31;
(b) a DNA that comprises a nucleotide sequence in which one or several nucleotides have been deleted, substituted or added in the nucleotide sequence set forth in SEQ ID NO: 29 or 31 and that has a function equivalent to that of a DNA comprising the nucleotide sequence set forth in SEQ ID NO: 29 in terms of the effect of reducing the anthocyanin content;
(c) a DNA that hybridizes to a complementary strand of a DNA comprising the nucleotide sequence set forth in SEQ ID NO: 29 or 31 under a stringent condition and that has a function equivalent to that of a DNA comprising the nucleotide sequence set forth in SEQ ID NO: 29 or 31 in terms of the effect of reducing the anthocyanin content; and
(d) a DNA that has a sequence identity of at least 90% with a DNA comprising the nucleotide sequence set forth in SEQ ID NO: 29 or 31 and that has a function equivalent to that of a DNA comprising the nucleotide sequence set forth in SEQ ID NO: 29 or 31 in terms of the effect of reducing the anthocyanin content.

[8] The method according to the above [7] wherein the expression of the above gene is suppressed by the RNAi method.

[9] A torenia obtained by the method described in the above [7] or [8], a progeny thereof having a property equivalent to said torenia in terms of the effect of reducing the anthocyanin content, or a tissue thereof.

[10] A gene comprising any of the following (a) to (d):
(a) a DNA comprising the nucleotide sequence set forth in SEQ ID NO: 29 or 31;
(b) a DNA that comprises a nucleotide sequence in which one or several nucleotides have been deleted, substituted or added in the nucleotide sequence set forth in SEQ ID NO: 29 or 31 and that has a function equivalent to that of a DNA comprising the nucleotide sequence set forth in SEQ ID NO: 29 in terms of the effect of reducing the anthocyanin content;
(c) a DNA that hybridizes to a complementary strand of a DNA comprising the nucleotide sequence set forth in SEQ ID NO: 29 or 31 under a stringent condition and that has a function equivalent to that of a DNA comprising the nucleotide sequence set forth in SEQ ID NO: 29 or 31 in terms of the effect of reducing the anthocyanin content; and
(d) a DNA that has a sequence identity of at least 90% with a DNA comprising the nucleotide sequence set forth in SEQ ID NO: 29 or 31 and that has a function equivalent to that of a DNA comprising the nucleotide sequence set forth in SEQ ID NO: 29 or 31 in terms of the effect of reducing the anthocyanin content.

[11] The method according to the above [2] for altering the depth of flower color of Ipomoea nil for the lighter by reducing the anthocyanin content in petals of Ipomoea nil, which comprises a step of suppressing the expression of a gene comprising any of the following (a) to (d):
(a) a DNA comprising the nucleotide sequence set forth in SEQ ID NO: 10;
(b) a DNA that comprises a nucleotide sequence in which one or several nucleotides have been deleted, substituted or added in the nucleotide sequence set forth in SEQ ID NO: 10 and that has a function equivalent to that of a DNA comprising the nucleotide sequence set forth in SEQ ID NO: 10 in terms of the effect of reducing the anthocyanin content;
(c) a DNA that hybridizes to a complementary strand of a DNA comprising the nucleotide sequence set forth in SEQ ID NO: 10 under a stringent condition and that has a function equivalent to that of a DNA comprising the nucleotide sequence set forth in SEQ ID NO: 10 in terms of the effect of reducing the anthocyanin content; and
(d) a DNA that has a sequence identity of at least 90% with a DNA comprising the nucleotide sequence set forth in SEQ ID NO: 10 and that has a function equivalent to that of a DNA comprising the nucleotide sequence set forth in SEQ ID NO: 10 in terms of the effect of reducing the anthocyanin content.

[12] The method according to the above [11] wherein the expression of the above gene is suppressed by the RNAi method.

[13] A gene comprising any of the following (a) to (d):
(a) a DNA comprising the nucleotide sequence set forth in SEQ ID NO: 10;
(b) a DNA that comprises a nucleotide sequence in which one or several nucleotides have been deleted, substituted or added in the nucleotide sequence set forth in SEQ ID NO: 10 and that has a function equivalent to that of a DNA comprising the nucleotide sequence set forth in SEQ ID NO: 10 in terms of the effect of reducing the anthocyanin content;
(c) a DNA that hybridizes to a complementary strand of a DNA comprising the nucleotide sequence set forth in

SEQ ID NO: 10 under a stringent condition and that has a function equivalent to that of a DNA comprising the nucleotide sequence set forth in SEQ ID NO: 10 in terms of the effect of reducing the anthocyanin content; and
(d) a DNA that has a sequence identity of at least 90% with a DNA comprising the nucleotide sequence set forth in SEQ ID NO: 10 and that has a function equivalent to that of a DNA comprising the nucleotide sequence set forth in SEQ ID NO: 10 in terms of the effect of reducing the anthocyanin content.

### Effect of the Invention

In accordance with the present invention, the depth of flower color can be altered for the lighter or deeper by varying the anthocyanin content in petals of the plant by regulating the expression of type IV chalcone isomerase.

### BRIEF EXPLANATION OF THE DRAWINGS

FIG. 1 shows the nucleotide sequence of a 254 bp DNA fragment that is specifically increased in the Chidori strain of Ipomoea nil.

FIG. 2 shows the result of RT-PCR analysis on the spatial expression of the CHI-B gene of Ipomoea nil.

FIG. 3 shows the result of Northern blot analysis of the transcription product of the CHI-B gene related to the developmental stage of the petal of Ipomoea nil.

FIG. 4 shows that the depth of flower color of the petal of a strain in which the amount of the transcription product of the PtCHI-B gene is decreased was altered for the lighter in petunia var. Surfinia purple mini (Suntory Flowers Limited).

FIG. 5 shows that the depth of flower color of the petal of a strain in which the amount of the transcription product of the TrCHI-B2 or TrCHI-B11 gene is decreased was altered for the lighter in torenia var. Summer wave blue (Suntory Flowers Limited).

### EMBODIMENTS FOR CARRYING OUT THE INVENTION

As described above, the function of type IV chalcone isomerase was unknown. It was now known either whether or not type IV chalcone isomerase was involved in the synthesis of anthocyanin nor were there any examples wherein the gene of type IV chalcone isomerase was used to alter flower color. Also, though a plurality of enzymes involved in the synthesis of anthocyanins have been postulated to be in a supermolecular structure due to interaction between proteins, it was not known that type IV chalcone isomerase was involved in such interaction.

The present invention is nucleotided in part on the finding that when the expression of type IV chalcone isomerase was suppressed, the anthocyanin content in petals of Ipomoea nil decreased thereby altering the depth of the flower color for the lighter.

As described above, both of type IV chalcone isomerases have no catalytic center of type I chalcone isomerase, and thus are considered to have no activity as the chalcone isomerase. Though not wishing to be bound by any specific theory, but the present inventors have assumed that type IV chalcone isomerase is involved in the formation of a supermolecular structure formed by interaction between a plurality of enzymes involved in the synthesis of anthocyanins, thereby affecting indirectly the anthocyanin content in the petal.

The first aspect of the present invention is a method for altering the depth of flower color of a plant for the lighter or deeper by varying the anthocyanin content in petals of the plant, which comprises a step of regulating the expression of type IV chalcone isomerase in the plant. Specifically, in accordance with the present invention, there is provided a method for altering the depth of flower color of a plant for the lighter by reducing the anthocyanin content in petals of the plant, which comprises a step of suppressing the expression of type IV chalcone isomerase in the plant. It would be possible to alter the depth of flower color of a plant for the deeper by overexpressing this gene.

As used herein, the term “type IV chalcone isomerase” indicates a chalcone isomerase that is classified into type IV according to the description in Nonpatent document 1.

Another aspect of the present invention is a method for altering the depth of flower color of the petunia plant for the lighter by reducing the anthocyanin content in petals of petunia, which comprises a step of suppressing the expression of a gene comprising any of the following (a) to (d):
(a) a DNA comprising the nucleotide sequence set forth in SEQ ID NO: 25;
(b) a DNA that comprises a nucleotide sequence in which one or several nucleotides have been deleted, substituted or added in the nucleotide sequence set forth in SEQ ID NO: 25 and that has a function equivalent to that of a DNA comprising the nucleotide sequence set forth in SEQ ID NO: 25 in terms of the effect of reducing the anthocyanin content;
(c) a DNA that hybridizes to a complementary strand of a DNA comprising the nucleotide sequence set forth in SEQ ID NO: 25 under a stringent condition and that has a function equivalent to that of a DNA comprising the nucleotide sequence set forth in SEQ ID NO: 25 in terms of the effect of reducing the anthocyanin content; and
(d) a DNA that has a sequence identity of at least 90% with a DNA comprising the nucleotide sequence set forth in SEQ ID NO: 25 and that has a function equivalent to that of a DNA comprising the nucleotide sequence set forth in SEQ ID NO: 25 in terms of the effect of reducing the anthocyanin content.

“One or several” in DNA of the above (b) means that 1-20, preferably 1-10, more preferably 1-5 nucleotides may be deleted, substituted or added in the nucleotide sequence set forth in SEQ ID NO: 25 as long as the DNA has a function equivalent to that of a DNA comprising the nucleotide sequence set forth in SEQ ID NO: 25 in terms of the effect of reducing the anthocyanin content. Also, “deletion”, “substitution” and “addition” refer to those that produce the nucleotide sequence encoding a protein having a property similar to the protein (SEQ ID NO: 26) encoded by the nucleotide sequence set forth in SEQ ID NO: 25.

The “stringent condition” in DNA of the above (c) may be established by deciding, as appropriate, the temperature and salt concentration during hybridization, preferably washing, depending on the DNA to be hybridized to a complementary strand of a DNA comprising the nucleotide sequence set forth in SEQ ID NO: 25. As the stringent condition, there can be mentioned those described in Sambrook et al., “Molecular Cloning: A Laboratory Manual 2nd Ed.” (Cold Spring Harbor Laboratory Press, 1989) and the like. Specifically, for example, there can be mentioned a condition in which (i) a DNA is incubated overnight at 42° C. with a probe in a solution containing 6×SSC (the composition of 1×SSC: 0.15 M NaCl, 0.015 M sodium citrate, pH 7.0), 0.5% SDS, 5×Denhardt's, 100 μg/ml denatured salmon sperm DNA and 50% formamide, and (ii) removing the nonspecifically hybridized probe by washing, wherein, with a view to enhancing precision, with a condition of lower ionic strength, for example 2×SSC, and more stringently 0.1×SSC, and/or a condition of a higher temperature, for example 40° C. lower, more stringently 30° C. lower, more stringently 25° C. lower, more stringently 10° C. lower than the Tm value of the nucleic acid to be used, specifically 25° C. or higher, more stringently 37° C. or higher, more stringently 42° C. or higher, more stringently 50° C. or higher, more stringently 60° C. or higher etc., though the temperature may vary depending on the Tm value of the nucleic acid to be used. Tm may be determined according to the following equation:

Tm=81.5+16.6(log[Na⁺])+0.41(% G+C)−(600/N),

wherein N represents the length of a nucleotide strand and % G+C represents the content of the guanine and cytosine residues in the oligonucleotide. Hybridization may be performed by referring to the above-mentioned textbook. The procedure as used herein may be carried out as appropriate by referring to said textbook.

As the hybridizable DNA, there can be mentioned a DNA that has a identity of at least 60% or higher, preferably 70% or higher, more preferably 80% or higher, still more preferably 90% or higher, more preferably 95% or higher, and most preferably 98% or higher with a DNA comprising the nucleotide sequence set forth in SEQ ID NO: 25 when calculated using an analysis software such as BLAST and FASTA. The above (d) defines a DNA that has a sequence identity of at least 90% with a DNA comprising the nucleotide sequence set forth in SEQ ID NO: 25 and that has a function equivalent to that of a DNA comprising the nucleotide sequence set forth in SEQ ID NO: 25 in terms of the effect of reducing the anthocyanin content.

The expression of the above gene may be suppressed by any of the antisense method, the sense method (cosuppression method), the RNAi method, and the like, preferably by the RNAi method.

A petunia obtained by the above method, a progeny thereof having a property equivalent to said petunia in terms of the effect of reducing the anthocyanin content, or a tissue and a part thereof, specifically a part containing the petal, are also within the scope of the present invention.

Also, a DNA etc. for use in the above method comprising the nucleotide sequence set forth in SEQ ID NO: 25 is also within the scope of the present invention.

Another aspect of the present invention is a method for altering the depth of flower color of a torenia plant for the lighter by reducing the anthocyanin content in petals of the torenia, which comprises a step of suppressing the expression of a gene comprising any of the following (a) to (d):
(a) a DNA comprising the nucleotide sequence set forth in SEQ ID NO: 29 or 31;
(b) a DNA that comprises a nucleotide sequence in which one or several nucleotides have been deleted, substituted or added in the nucleotide sequence set forth in SEQ ID NO: 29 or 31 and that has a function equivalent to that of a DNA comprising the nucleotide sequence set forth in SEQ ID NO: 29 in terms of the effect of reducing the anthocyanin content;
(c) a DNA that hybridizes to a complementary strand of a DNA comprising the nucleotide sequence set forth in SEQ ID NO: 29 or 31 under a stringent condition and that has a function equivalent to that of a DNA comprising the nucleotide sequence set forth in SEQ ID NO: 29 or 31 in terms of the effect of reducing the anthocyanin content; and
(d) a DNA that has a sequence identity of at least 90% with a DNA comprising the nucleotide sequence set forth in SEQ ID NO: 29 or 31 and that has a function equivalent to that of a DNA comprising the nucleotide sequence set forth in SEQ ID NO: 29 or 31 in terms of the effect of reducing the anthocyanin content.

A still another aspect of the present invention is a method for altering the depth of flower color of Ipomoea nil for the lighter by reducing the anthocyanin content in petals of Ipomoea nil, which comprises a step of suppressing the expression of a gene comprising any of the following (a) to (d):
(a) a DNA comprising the nucleotide sequence set forth in SEQ ID NO: 10;
(b) a DNA that comprises a nucleotide sequence in which one or several nucleotides have been deleted, substituted or added in the nucleotide sequence set forth in SEQ ID NO: 10 and that has a function equivalent to that of a DNA comprising the nucleotide sequence set forth in SEQ ID NO: 10 in terms of the effect of reducing the anthocyanin content;
(c) a DNA that hybridizes to a complementary strand of a DNA comprising the nucleotide sequence set forth in SEQ ID NO: 10 under a stringent condition and that has a function equivalent to that of a DNA comprising the nucleotide sequence set forth in SEQ ID NO: 10 in terms of the effect of reducing the anthocyanin content; and
(d) a DNA that has a sequence identity of at least 90% with a DNA comprising the nucleotide sequence set forth in SEQ ID NO: 10 and that has a function equivalent to that of a DNA comprising the nucleotide sequence set forth in SEQ ID NO: 10 in terms of the effect of reducing the anthocyanin content.

The above description regarding DNA comprising the nucleotide sequence set forth in SEQ ID NO: 25 may also apply to a DNA comprising a nucleotide sequence set forth in any of SEQ ID NO: 29, 31 and 10, and the expression of the above genes may be suppressed by any of the antisense method, the sense method (cosuppression method), the RNAi method, and the like, preferably by the RNAi method. The torenia or Ipomoea nil, or a progeny thereof having a property equivalent to said torenia or Ipomoea nil in terms of the effect of reducing the anthocyanin content, or a tissue and a part thereof, specifically a part containing the petal, are also within the scope of the present invention. Furthermore, a DNA etc. for use in the above method comprising the nucleotide sequence set forth in SEQ ID NO: 29, 31 and 10 is also within the scope of the present invention.

Hereinbelow, the present invention will now be specifically explained with respect to Examples, but it should recognized that the present invention is limited to them in any way.

### EXAMPLES

### Example 1

### Obtaining the DNA Fragment of Ipomoea Nil

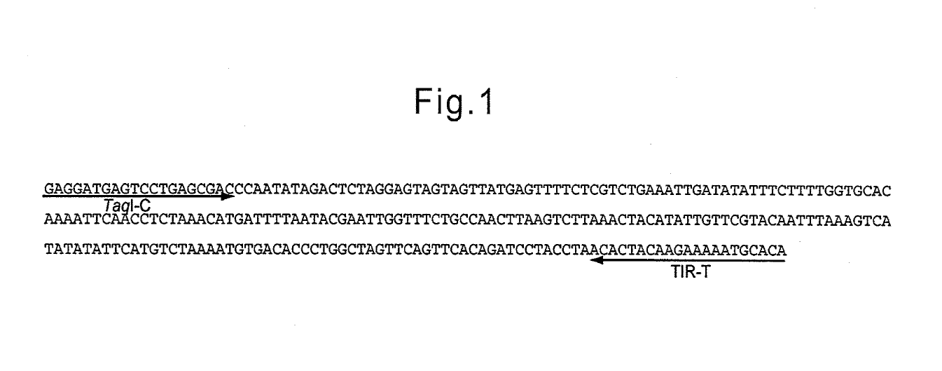

The present inventors have assumed that the genetic difference between the Chidori strain that blooms red flowers and the Kusumi Chidori strain that blooms pink flowers was derived from the transposon inserted into the causative gene. Thus, using the transposon display method (see Fukada-Tanaka et al. Plant Biotech. 18, 143-149, 2001), a transposon-adjacent region was amplified, and a 259 bp DNA fragment that was specifically increased in the Kusumi Chidori strain was obtained (SEQ ID NO: 1, see FIG. 1). For amplification of the DNA fragment, after the Taq1 adapter (5′-GCGGATGAGTCCTGAG-3′ (SEQ ID NO: 25) and 5′-CGCTCAGGACTCAT-3′ (SEQ ID NO: 3)) was bound to the genomic DNA digested with a restriction enzyme Taq1, two primers of Tqq1-C (5′-GAGGATGAGTCCTGAGCGAC-3′, SEQ ID NO: 4) and TIR-T (5′-TGTGCATTTTTCTTGTAGTGT-3′, SEQ ID NO: 5) were used. Since the DNA fragment obtained by the transposon display method did not code any protein, an adjacent genomic region was isolated from the red Chidori strain by the inverse PCR method. For amplification of the DNA fragment, after the genomic DNA digested with a restriction enzyme Taq1 was cyclized using T4 DNA ligase, PCR was carried out on the DNA fragment obtained by the transposon display method using the designed primer. For primary amplification, two types of primers of IPCR-A1 (5′-ATTGATGTCTAAAATGTGACACC-3′, SEQ ID NO: 6) and IPCR-B1 (5′-TCAATTTCAGACGAGAAAACTC-3′, SEQ ID NO: 7) were used, and for secondary amplification, two types of primers of IPCR-A2 (5′-TGACACCCTGGCTAGTTCAG-3′, SEQ ID NO: 8) and IPCR-B2 (5′-GAAAACTCATAACTACTACTCC-3′, SEQ ID NO: 9) were used. The 1.1 kb DNA region obtained by the inverse PCR method exhibited homology with a chalcone isomerase (AY595417) (see Nonpatent document 1) that is classified into type IV isolated from soy bean.

### Example 2

### Gene Screening

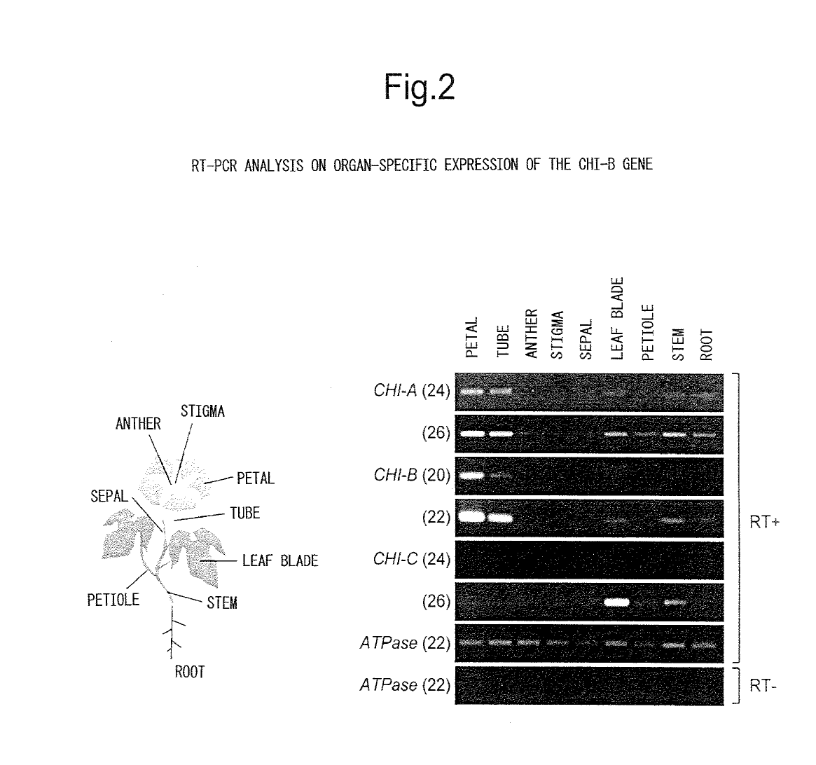

From the database of the EST library created using RNA extracted from the buds and seedlings of the wild type Ipomoea nil strain TKS in the National BioResource Project (NBRP), cDNA corresponding to the above 1.1 kb DNA region obtained by the inverse PCR method was isolated, and the gene was designated as CHI-B. The sequence is shown in SEQ ID NO: 10. The amino acid sequence (SEQ ID NO: 11) encoded by this sequence had an identity of 59% and 56% with the above-mentioned Arabidopsis AT5G05270 and soy bean AY595417, respectively. The tissue-specificity of the expression of the gene was investigated by RT-PCR. RNA obtained from each tissue by the CsCl method or the Get pureRNA Kit (Dojindo Laboratories) was retranscribed to DNA using the SuperScript First Strand System (for RT-PCR) (Invitrogen), and, with the DNA as the template, PCR was conducted using CHI-B LA-F2 (5′-AGTTCTTCTTGCAGGCTGCAGAC-3′, SEQ ID NO: 12) and CHI-B R1 (5′-ACTCCATAGGATCACCAAACTCTC-3′, SEQ ID NO: 13) as the primer, the result of which is shown in FIG. 2. It can been seen from FIG. 2 that the CHI-B gene is specifically expressed in the petal. Furthermore, in FIG. 2, CHI-A and CHI-C represent the gene encoding type 1 chalcone isomerase and type III chalcone isomerase, respectively.

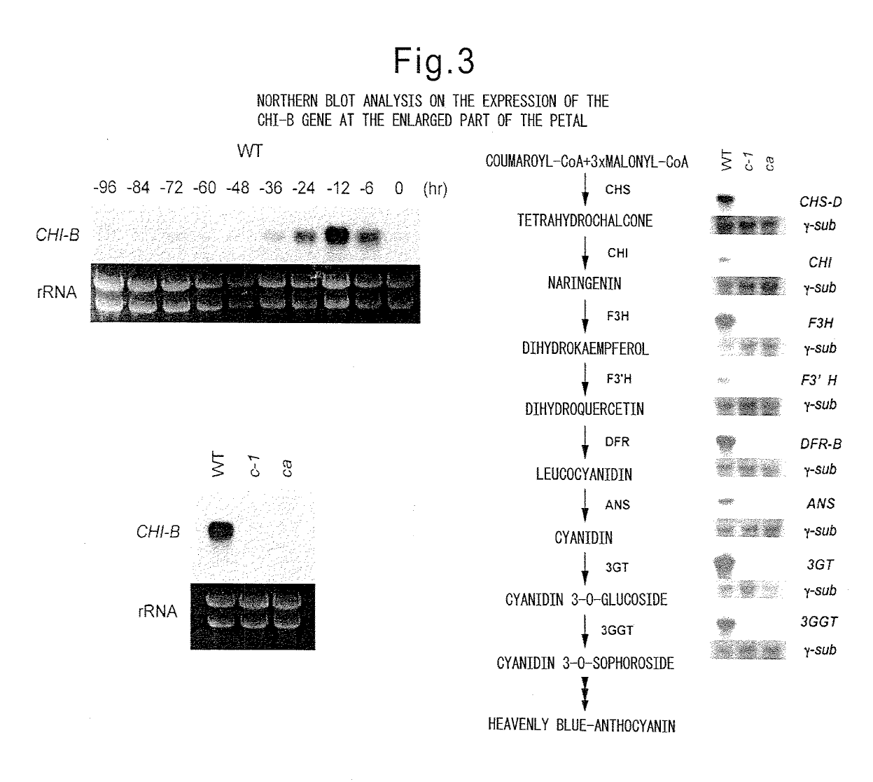

Northern blot analysis of the transcription product of the CHI-B gene depending on the developmental stage of the petal revealed that it is most highly expressed at 12 hours before flowering as shown in FIG. 3. In the petals of the flowers in which the transcription control factor c-1 or ca is mutated, no expression of the CHI-B gene was observed. These results suggest that CHI-B plays a role in the synthesis of anthocyanins.

### Example 3

### Obtaining and Characterizing the Function of the Homolog of Petunia

Based on the amino acid sequences that are not conserved in the CHI-A protein but conserved in the CHI-B protein, the following primers were synthesized, in which M represents deoxyinosine, H represents a mixture of A, T and C, Y represents a mixture of C and T, R represents a mixture of A and G, B represents deoxyuracil, W represents a mixture of A and T, D represents a mixture of A and T, K represents a mixture of T and G, and S represents a mixture of C and G.

CHIB-F1 (5′-GGMATHACMGAYGTMGARABHCAYTTY-3′,
SEQ ID NO: 14)
CHIB-F2 (5′-TMWSMYTMHTMGGMCAMGGMATHAC-3′,
SEQ ID NO: 15)
CHI-B R1 (5′-CCYTTDATYTCYTMDATMACMAC-3′,
SEQ ID NO: 16)
CHI-B R2 (5′-TCYTCYTCYTCYTCRTAYTTKTC-3′,
SEQ ID NO: 17)
CHI-B R3 (5′-TTYTTRAARTAYTTMSWYTGRAA-3′,
SEQ ID NO: 18)

From the buds of a petunia, RNA was extracted using the RNeasy Plant Kit (QIAGEN). Using the SuperScript First Strand system (Invitrogen), cDNA was synthesized. With the cDNA as the template, PCR was carried out using the primer set of (CHIB-F1, CHI-B R1), (CHIB-F1, CHI-B R2), (CHIB-F1, CHI-B R3), (CHIB-F2, CHI-B R1), (CHIB-F2, CHI-B R2), and (CHIB-F2, CHI-B R3). The reaction was conducted in 25 μl for 30 cycles with one cycle comprising 95° C. for 30 sec, 72° C. for 30 sec, and 55° C. for 30 sec. Each reaction mixture was diluted 100-fold, and 1 μl thereof was used the template to repeat the PCR as described above. As a result, a DNA band was obtained with the primer set of (CHIB-F2, CHI-B R2). This was ligated to pCR2.1 TOPO to obtain pSPB3387 in which partial cDNA of CHI-B had been inserted.

In order to obtain a full-length cDNA, the sequences of the 5′-end and the 3′-end of the above DNA fragment were amplified using the Gene Racer (Invitrogen) according to the protocol recommended by the manufacturer. Thus, for the sequence of the 5′ end, the first PCR was conducted with the cDNA of the petal as the template using the GeneRacer 5′ primer (5′-CGACTGGAGCACGAGGACACTGA-3′, SEQ ID NO: 19) and the pCHIB-R1 primer (5′-CTCTTACAGCACTCTCTAGC-3′, SEQ ID NO: 20). With the amplified DNA as the template, a second PCR was conducted using the GeneRacer 5′ nested primer (5′-GGACACTGACATGGACTGAAGGAGTA-3′, SEQ ID NO: 21) and the PtCHIB-R1 primer. This DNA band was ligated to pCR2.1 TOPO to obtain pSPB3401. For the sequence of the 3′-end, the first PCR was conducted with the cDNA of the petal as the template using the GeneRacer 3′ primer (5′-GCTGTCAACGATACGCTACGTAACG-3′, SEQ ID NO: 22) and the PtCHIB-R1 primer (5′-CATTGAGATACACTTTCTCC-3′, SEQ ID NO: 23). With the amplified DNA as the template, a second PCR was conducted using the GeneRacer 3′ nested primer (5′-CGCTACGTAACGGCATGACAGTG-3′, SEQ ID NO: 24) and the PtCHIB-F1 primer. This DNA band was ligated to pCR2.1 TOPO to obtain pSPB3402. By ligating a DNA fragment obtained by digesting pSPB3401 with BamHI to pSPB3402 obtained by digesting with BamHI, a plasmid pSPB3400 containing the full-length (SEQ ID NO: 25) of petunia CHI-B classified into type IV chalcone isomerase was obtained. An amino acid sequence (SEQ ID NO: 26) encoded by this sequence exhibited an identity of 74%, 56% and 58% with Ipomoea nil CHI-B, Arabidopsis AT5G05270 and soy bean AY595417, respectively.

A plasmid pSPB3407 was obtained by ligating (i) a DNA fragment obtained by digesting pSPB3401 with EcoRV and XhoI, (ii) a DNA fragment obtained by digesting pSPB3401 with XhoI and BamHI, and (iii) an about 400 bp DNA fragment obtained by digesting pSPB3400 with BamHI and RsaI. This plasmid was digested with KpnI and XbaI to recover an about 1050 bp DNA fragment, which was ligated to a binary vector digested with XbaI and KpnI. The plasmid obtained was designated as pSPB3408. This plasmid was intended to transcribe constitutively the double stranded RNA of the CHI-B gene of petunia.

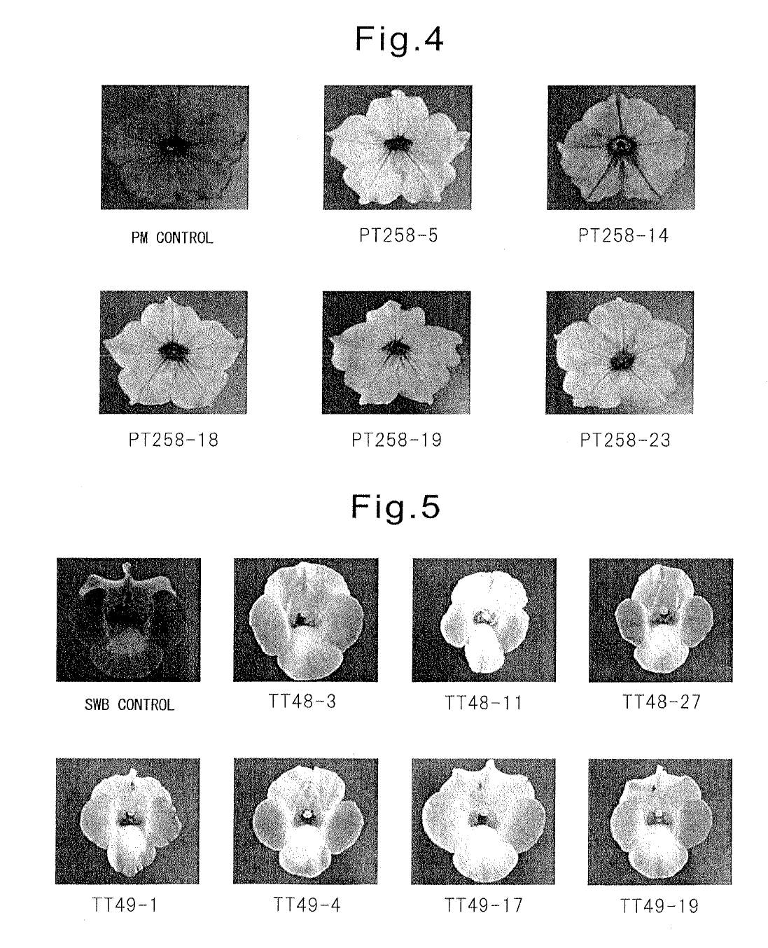

In order to suppress the expression of the PtCHI-B gene, petunia var. Surfinia purple mini (Suntory Flowers Co., Ltd.) was transformed with an Agrobacterium containing a binary vector pSPB3408. From petals of the buds of the transformant obtained, RNA was extracted, and the amount of the transcription product of the PtCHI-B gene contained in petals of the buds of the transformant obtained was determined using the RT-PCR kit (Promega, PtCHIB-FW (5′-ATGGGAAAGAACGAAGTGATGG-3′, SEQ ID NO: 27) and PtCHIB-RV (5′-TCATTTAGATAATTCAGCAGAG-3′, SEQ ID NO: 28) as the primer). The petals of the strain in which the amount of the transcription product was decreased had a lighter depth of flower color than the petals of the host or the strain in which the amount of the transcription product was not decreased (see FIG. 4). From 0.5 g of these petals, anthocyanin was extracted, and after hydrolysis, the amount of anthocyanidine was determined by high performance liquid chromatography. The actual procedure was carried out as described in WO2005/017147. The relative amount of the total anthocyanidine was compared using the peak area obtained by high performance liquid chromatography as the index. The area of the petunia in which CHI-B was suppressed decreased to about 30%. The result is shown in the following Table 1.

TABLE 1

Anthocyanidine

| Sample name | Total area | (%) |
|---|---|---|
| PT258-5 | 1010044 | 28.80 |
| PT258-18 | 1022113 | 29.14 |
| PT258-19 | 857051 | 24.44 |
| PT258-23 | 1043910 | 29.77 |
| PM control | 3507076 | 100.00 |

Table 1 and FIG. 4 indicated that by suppressing the expression of the CHI-B gene of petunia the anthocyanin content in the petal decreased and the depth of flower color was altered for the lighter.

### Example 4

### Obtaining and Characterizing the Function of the Homolog of Torenia

In a similar manner to Example 3, the homolog of torenia was obtained. From the petals of the buds of torenia var. Summer wave blue (Suntory Flowers Ltd.), RNA was extracted. Using this as the template, cDNA was synthesized, and PCR was carried out using the primers CHIB-F2 and CHI-B R2. The DNA fragment obtained was cloned into pCRTOPO, which was designated as pSPB3388. Using this, a cDNA library of torenia (described in Molecular Breeding 6, 239-246, 2000) was screened, and TrCHI-B2 (SEQ ID NO: 29) and TrCHI-B11 (SEQ ID NO: 31) were obtained as the genes encoding proteins classified into type IV chalcone isomerase. Plasmids pSPB3398 and pSPB3399 containing each of them were obtained. The amino acid sequence (SEQ ID NO: 30) encoded by SEQ ID NO: 29 had an identity of 67%, 70%, 58% and 57% with Ipomoea nil CHI-B, petunia CHI-B, Arabidopsis AT5G05270 and soy bean AY595417, respectively. The amino acid sequence (SEQ ID NO: 32) encoded by SEQ ID NO: 31 had an identity of 65%, 70%, 94%, 58% and 57% with Ipomoea nil CHI-B, petunia CHI-B, TrCHI-B2, Arabidopsis AT5G05270 and soy bean AY595417, respectively.

A DNA fragment obtained by digesting plasmid pSFL313 described in WO2005/059141 with BamHI, a 540 bp DNA fragment obtained by digesting pSPB3398 with BglII and SmaI, and a 280 bp DNA fragment obtained by digesting pSPB3398 with BglII and PvuII were ligated to obtain pSPB3403. A DNA fragment obtained by digesting this plasmid with HindIII and EcoRI was cloned into HindIII and EcoRI of pBIn+. This binary vector pSPB3405 is intended to transcribe constitutively the double stranded RNA of torenia CHI-B2.

A binary vector for suppressing the expression of ThCHI-B11 was created as follows. A DNA fragment obtained by digesting plasmid pSFL313 with BamHI, a 670 bp DNA fragment obtained by digesting pSPB3399 with BglII and HindIII, and a 360 bp DNA fragment obtained by digesting pSPB3399 with BglII and EcoRI were ligated to obtain pSPB3404. A DNA fragment obtained by digesting this plasmid with AscI was cloned into AscI of pBIn+. This binary vector pSPB3406 is intended to transcribe constitutively the double stranded RNA of torenia CHI-B11.

Torenia var. Summer wave blue (Suntory Flowers Ltd.) was transformed with an Agrobacterium containing the binary vector pSPB3405. From the petals of the buds of the transformant obtained, RNA was extracted. The amount of the transcription product of TrCHI-B2 contained in petals of the buds of the transformant obtained was determined using the RT-PCR kit (Promega, primer TrCHIB-FW (5′-GTATGGCCGGCGGTGAAG-3′, SEQ ID NO: 33) and primer TrCHIB-RV (5^{v}-CTCATTTCGATAACTCAGC-3′, SEQ ID NO: 34)). The petals of the strain in which the amount of the transcription product was decreased had a lighter depth of flower color than the petals of the host or the strains in which the amount of the transcription product was not decreased (see FIG. 5, upper part, Table 2). By soaking 0.5 g of these petals in 5 ml of methanol containing 1% hydrochloric acid, anthocyanin was extracted. The spectrum of the anthocyanin extracted at 400 nm to 500 nm was measured. When anthocyanin was determined from the absorbance at the maximum wavelength, the amount of anthocyanin in petals of the strains in which the amount of the transcription products was decreased was smaller.

TABLE 2
The amount of anthocyanin in torenia in which TrCHIB-2 was suppressed

| | 1 petal* | 5 petals* | |
|---|---|---|---|
| | Abs | Abs | |
| SWB control 1 | 3.2964 | 3.0257 | 2.9437 |
| SWB control 2 | 2.4779 | 2.9118 | |
| SWB control 3 | | 2.9558 | |
| SWB control 4 | | 2.8815 | |
| TT48-01 | 1.7722 | 2.4328 | 83% |
| TT48-03 | 1.3676 | 1.5442 | 52% |
| TT48-04 | 2.1590 | 2.2286 | 76% |
| TT48-05 | 1.9636 | 1.8974 | 64% |
| TT48-06 | 2.6686 | 2.6387 | 90% |
| TT48-07 | 2.2183 | 1.7274 | 59% |
| TT48-08 | 1.8605 | 1.7371 | 59% |
| TT48-10 | 3.0703 | 2.4479 | 83% |
| TT48-11 | 1.3214 | 1.6277 | 55% |
| TT48-13 | 1.8553 | 2.1544 | 73% |
| TT48-15 | 1.8082 | 1.5526 | 53% |
| TT48-16 | 1.5370 | 1.9595 | 67% |
| TT48-17 | 1.9874 | 1.7049 | 58% |
| TT48-18 | 1.6954 | 1.5199 | 52% |
| TT48-19 | 2.0946 | 1.7944 | 61% |
| TT48-20 | 1.9380 | 2.1621 | 73% |
| TT48-22 | 1.9738 | 1.7237 | 59% |
| TT48-24 | 1.8430 | 1.7802 | 60% |
| TT48-27 | 1.6287 | 1.4386 | 49% |
| TT48-28 | 1.5888 | 1.7028 | 58% |

*1 petal and 5 petals indicate the values obtained by extracting one petal and five petals at one time, respectively.
The relative value (%) to 100% of the control flower is shown in the rightmost column.

In a similar manner, torenia var. Summer wave blue (Suntory Flowers Ltd.) was transformed with an Agrobacterium containing a binary vector pSPB3406. From the petals of the buds of the transformant obtained, RNA was extracted, and the amount of the transcription product of TrCHI-B11 contained in petals of the buds of the transformant obtained was determined using the RT-PCR kit (manufacturer, primer). The petals of the strain in which the amount of the transcription product was decreased had a lighter depth of flower color than the petals of the host or the strains in which the amount of the transcription product was not decreased (see FIG. 5, bottom part, Table 3). By soaking 0.5 g of these petals in 5 ml of methanol containing 1% hydrochloric acid, anthocyanin was extracted. The spectrum of the extracted anthocyanin was measured at 400 nm to 500 nm. When anthocyanin was determined from the absorbance at the maximum wavelength, the amount of anthocyanin in petals of the strains in which the amount of the transcription products was decreased was smaller.

TABLE 3
The amount of anthocyanin in torenia in which TrCHIB-1 was suppressed

| | 1 petal* | 5 petals* | |
|---|---|---|---|
| | Abs | Abs | |
| SWB control 1 | 2.8617 | 2.6345 | |
| SWB control 2 | 2.9522 | | |
| TT49-01 | 1.5107 | 1.2657 | 48% |
| TT49-02 | 1.8885 | 1.2744 | 48% |
| TT49-03 | 1.6459 | 1.3020 | 49% |
| TT49-04 | 1.3035 | 1.3038 | 49% |
| TT49-05 | 1.7244 | 1.4796 | 56% |
| TT49-06 | 1.9887 | 1.4151 | 54% |
| TT49-08 | 2.3415 | 1.5693 | 60% |
| TT49-09 | 1.7536 | 1.6736 | 64% |
| TT49-14 | 1.7175 | 1.4984 | 57% |
| TT49-15 | 2.1824 | 1.2734 | 48% |
| TT49-17 | 1.5029 | 1.2517 | 48% |
| TT49-19 | 1.4543 | 1.2174 | 46% |
| TT49-20 | 1.8943 | 1.5268 | 58% |
| TT49-22 | 1.7928 | 1.4137 | 54% |
| TT49-23 | 1.5499 | 1.5955 | 61% |
| TT49-29 | 2.1604 | 1.4984 | 57% |

*1 petal and 5 petals indicate the values obtained by extracting one petal and five petals at one time, respectively.
The relative value (%) to 100% of the control flower is shown in the rightmost column.

### INDUSTRIAL APPLICABILITY

The present invention can provide a method for altering the depth of flower color of a plant for the lighter by reducing the anthocyanin content in petals of the plant, which comprises a step of suppressing the expression of type IV chalcone isomerase in the plant, and a novel gene useful in said method, and therefore may preferably be used in the flower industry.

1. A method for altering the depth of flower color for the lighter or deeper by varying the anthocyanin content in petals of a plant, which comprises a step of regulating the expression of type IV chalcone isomerase in the plant.

2. A method for altering the depth of flower color for the lighter by reducing the anthocyanin content in petals of a plant, which comprises a step of suppressing the expression of type IV chalcone isomerase in the plant.

3. The method according to claim 2 for altering the depth of flower color of a petunia plant for the lighter by reducing the anthocyanin content in petals of the petunia, which comprises a step of suppressing the expression of a gene comprising any of the following (a) to (d):
(a) a DNA comprising the nucleotide sequence set forth in SEQ ID NO: 25;
(b) a DNA that comprises a nucleotide sequence in which one or several nucleotides have been deleted, substituted or added in the nucleotide sequence set forth in SEQ ID NO: 25 and that has a function equivalent to that of a DNA comprising the nucleotide sequence set forth in SEQ ID NO: 25 in terms of the effect of reducing the anthocyanin content;
(c) a DNA that hybridizes to a complementary strand of a DNA comprising the nucleotide sequence set forth in SEQ ID NO: 25 under a stringent condition and that has a function equivalent to that of a DNA comprising the nucleotide sequence set forth in SEQ ID NO: 25 in terms of the effect of reducing the anthocyanin content; and
(d) a DNA that has a sequence identity of at least 90% with a DNA comprising the nucleotide sequence set forth in SEQ ID NO: 25 and that has a function equivalent to that of a DNA comprising the nucleotide sequence set forth in SEQ ID NO: 25 in terms of the effect of reducing the anthocyanin content.

4. The method according to claim 3 wherein the expression of said gene is suppressed by the RNAi method.

5. A petunia obtained by the method described in claim 3, a progeny thereof having a property equivalent to said petunia in terms of the effect of reducing the anthocyanin content, or a tissue thereof.

6. A gene comprising any of the following (a) to (d):
(a) a DNA comprising the nucleotide sequence set forth in SEQ ID NO: 25;
(b) a DNA that comprises a nucleotide sequence in which one or several nucleotides have been deleted, substituted or added in the nucleotide sequence set forth in SEQ ID NO: 25 and that has a function equivalent to that of a DNA comprising the nucleotide sequence set forth in SEQ ID NO: 25 in terms of the effect of reducing the anthocyanin content;
(c) a DNA that hybridizes to a complementary strand of a DNA comprising the nucleotide sequence set forth in SEQ ID NO: 25 under a stringent condition and that has a function equivalent to that of a DNA comprising the nucleotide sequence set forth in SEQ ID NO: 25 in terms of the effect of reducing the anthocyanin content; and
(d) a DNA that has a sequence identity of at least 90% with a DNA comprising the nucleotide sequence set forth in SEQ ID NO: 25 and that has a function equivalent to that of a DNA comprising the nucleotide sequence set forth in SEQ ID NO: 25 in terms of the effect of reducing the anthocyanin content.

7. The method according to claim 2 for altering the depth of flower color of the torenia plant for the lighter by reducing the anthocyanin content in petals of the torenia, which comprises a step of suppressing the expression of a gene comprising any of the following (a) to (d):
(a) a DNA comprising the nucleotide sequence set forth in SEQ ID NO: 29 or 31;
(b) a DNA that comprises a nucleotide sequence in which one or several nucleotides have been deleted, substituted or added in the nucleotide sequence set forth in SEQ ID NO: 29 or 31 and that has a function equivalent to that of a DNA comprising the nucleotide sequence set forth in SEQ ID NO: 29 in terms of the effect of reducing the anthocyanin content;
(c) a DNA that hybridizes to a complementary strand of a DNA comprising the nucleotide sequence set forth in SEQ ID NO: 29 or 31 under a stringent condition and that has a function equivalent to that of a DNA comprising the nucleotide sequence set forth in SEQ ID NO: 29 or 31 in terms of the effect of reducing the anthocyanin content; and
(d) a DNA that has a sequence identity of at least 90% with a DNA comprising the nucleotide sequence set forth in SEQ ID NO: 29 or 31 and that has a function equivalent to that of a DNA comprising the nucleotide sequence set forth in SEQ ID NO: 29 or 31 in terms of the effect of reducing the anthocyanin content.

8. The method according to claim 7 wherein the expression of said gene is suppressed by the RNAi method.

9. A torenia obtained by the method described in claim 7, a progeny thereof having a property equivalent to said torenia in terms of the effect of reducing the anthocyanin content, or a tissue thereof.

10. A gene comprising any of the following (a) to (d):
(a) a DNA comprising the nucleotide sequence set forth in SEQ ID NO: 29 or 31;
(b) a DNA that comprises a nucleotide sequence in which one or several nucleotides have been deleted, substituted or added in the nucleotide sequence set forth in SEQ ID NO: 29 or 31 and that has a function equivalent to that of a DNA comprising the nucleotide sequence set forth in SEQ ID NO: 29 in terms of the effect of reducing the anthocyanin content;
(c) a DNA that hybridizes to a complementary strand of a DNA comprising the nucleotide sequence set forth in SEQ ID NO: 29 or 31 under a stringent condition and that has a function equivalent to that of a DNA comprising the nucleotide sequence set forth in SEQ ID NO: 29 or 31 in terms of the effect of reducing the anthocyanin content; and
(d) a DNA that has a sequence identity of at least 90% with a DNA comprising the nucleotide sequence set forth in SEQ ID NO: 29 or 31 and that has a function equivalent to that of a DNA comprising the nucleotide sequence set forth in SEQ ID NO: 29 or 31 in terms of the effect of reducing the anthocyanin content.

11. The method according to claim 2 for altering the depth of flower color of Ipomoea nil for the lighter by reducing the anthocyanin content in petals of Ipomoea nil, which comprises a step of suppressing the expression of a gene comprising any of the following (a) to (d):
(a) a DNA comprising the nucleotide sequence set forth in SEQ ID NO: 10;
(b) a DNA that comprises a nucleotide sequence in which one or several nucleotides have been deleted, substituted or added in the nucleotide sequence set forth in SEQ ID NO: 10 and that has a function equivalent to that of a DNA comprising the nucleotide sequence set forth in SEQ ID NO: 10 in terms of the effect of reducing the anthocyanin content;
(c) a DNA that hybridizes to a complementary strand of a DNA comprising the nucleotide sequence set forth in SEQ ID NO: 10 under a stringent condition and that has a function equivalent to that of a DNA comprising the nucleotide sequence set forth in SEQ ID NO: 10 in terms of the effect of reducing the anthocyanin content; and
(d) a DNA that has a sequence identity of at least 90% with a DNA comprising the nucleotide sequence set forth in SEQ ID NO: 10 and that has a function equivalent to that of a DNA comprising the nucleotide sequence set forth in SEQ ID NO: 10 in terms of the effect of reducing the anthocyanin content.

12. The method according to claim 11 wherein the expression of said gene is suppressed by the RNAi method.

13. A gene comprising any of the following (a) to (d):
(a) a DNA comprising the nucleotide sequence set forth in SEQ ID NO: 10;
(b) a DNA that comprises a nucleotide sequence in which one or several nucleotides have been deleted, substituted or added in the nucleotide sequence set forth in SEQ ID NO: 10 and that has a function equivalent to that of a DNA comprising the nucleotide sequence set forth in SEQ ID NO: 10 in terms of the effect of reducing the anthocyanin content;
(c) a DNA that hybridizes to a complementary strand of a DNA comprising the nucleotide sequence set forth in SEQ ID NO: 10 under a stringent condition and that has a function equivalent to that of a DNA comprising the nucleotide sequence set forth in SEQ ID NO: 10 in terms of the effect of reducing the anthocyanin content; and
(d) a DNA that has a sequence identity of at least 90% with a DNA comprising the nucleotide sequence set forth in SEQ ID NO: 10 and that has a function equivalent to that of a DNA comprising the nucleotide sequence set forth in SEQ ID NO: 10 in terms of the effect of reducing the anthocyanin content.

14. A petunia obtained by the method described in claim 4, a progeny thereof having a property equivalent to said petunia in terms of the effect of reducing the anthocyanin content, or a tissue thereof.

15. A torenia obtained by the method described in claim 8, a progeny thereof having a property equivalent to said torenia in terms of the effect of reducing the anthocyanin content, or a tissue thereof.


SEQUENCE LISTING (34 sequences)

SEQ ID NO: 1 - DNA, 259 bp, Ipomea nil
  Kusumi Chidori
gaggatgagtcctgagcgacccaatatagactctaggagtagtagttatgagttttctcgtctgaaattgatatatttcttttggtgcacaaaattcaacctctaaacatgattttaatacgaattggtttctgccaacttaagtcttaaactacatattgttcgtacaatttaaagtcatatatattcatgtctaaaatgtgacaccctggctagttcagttcacagatcctacctaacactacaagaaaaatgcaca

SEQ ID NO: 2 - DNA, 16 bp, Artificial Sequence
  Description of Artificial Sequence Synthetic TaqI adapter oligonucleotide
gcggatgagtcctgag

SEQ ID NO: 3 - DNA, 14 bp, Artificial Sequence
  Description of Artificial Sequence Synthetic TaqI adapter oligonucleotide
cgctcaggactcat

SEQ ID NO: 4 - DNA, 20 bp, Artificial Sequence
  Description of Artificial Sequence Synthetic Tqq1-C primer
gaggatgagtcctgagcgac

SEQ ID NO: 5 - DNA, 21 bp, Artificial Sequence
  Description of Artificial Sequence Synthetic TIR-T primer
tgtgcatttttcttgtagtgt

SEQ ID NO: 6 - DNA, 23 bp, Artificial Sequence
  Description of Artificial Sequence Synthetic IPCR-A1 primer
attcatgtctaaaatgtgacacc

SEQ ID NO: 7 - DNA, 22 bp, Artificial Sequence
  Description of Artificial Sequence Synthetic IPCR-B1 primer
tcaatttcagacgagaaaactc

SEQ ID NO: 8 - DNA, 20 bp, Artificial Sequence
  Description of Artificial Sequence Synthetic IPCR-A2 primer
tgacaccctggctagttcag

SEQ ID NO: 9 - DNA, 22 bp, Artificial Sequence
  Description of Artificial Sequence Synthetic IPCR-B2 primer
gaaaactcataactactactcc

SEQ ID NO: 10 - DNA, 621 bp, Ipomoea nil
  CHI-B
atgggtactgaaatggtgatggtggatgaaatcccatttcctccccaggtcaatctggacaataagctgctatcactgatgggtcatgggattactgatgttgagatacattttctccaaataaagtacactgcaattggagtttatttggacccagaaattgtgtcacatttgcagaaatggaagggcaaaacaccagttgacctagctcaagatgatgatttctttgaggctattatcaatgctccagtggataaagtgttgagagtggtggtgattaaggagatcaaagggtcacagtatggggtgcagcttgagaattctgtgagggacctattggcagaggtggataaatatgaagaggaagaggaagctgcccttgagaaagttgtggactttttccagtccaaatatttcaagaaaagttctgtcatcacattttctttcccagccaacactgccacagctaagattgtgtttgccacggaggggaaggaggactcatcaattgaagtggagaatgcaaatgtgggtgggatgatcaagaaatggtatttgggtggaagcagggcagtatcccctagtacaatctcatccttagccaacatactgcctgactaa

SEQ ID NO: 11 - protein, 206 aa, Ipomoea nil
MGTEMVMVDEIPFPPQVNLDNKLLSLMGHGITDVEIHFLQIKYTAIGVYLDPEIVSHLQKWKGKTPVDLAQDDDFFEAIINAPVDKVLRVVVIKEIKGSQYGVQLENSVRDLLAEVDKYEEEEEAALEKVVDFFQSKYFKKSSVITFSFPANTATAKIVFATEGKEDSSIEVENANVGGMIKKWYLGGSRAVSPSTISSLANILPD

SEQ ID NO: 12 - DNA, 23 bp, Artificial Sequence
  Description of Artificial Sequence Synthetic CHI-B LA-F2 primer
agttcttcttgcaggctgcagac

SEQ ID NO: 13 - DNA, 24 bp, Artificial Sequence
  Description of Artificial Sequence Synthetic CHI-B R1 primer
actccataggatcaccaaactctc

SEQ ID NO: 14 - DNA, 27 bp, Artificial Sequence
  Description of Artificial Sequence Synthetic CHIB-F1 primer
ggnathacngaygtngaranhcaytty

SEQ ID NO: 15 - DNA, 25 bp, Artificial Sequence
  Description of Artificial Sequence Synthetic CHIB-F2 primer
tnwsnytnhtnggncanggnathac

SEQ ID NO: 16 - DNA, 23 bp, Artificial Sequence
  Description of Artificial Sequence Synthetic CHI-B R1 primer
ccyttdatytcytndatnacnac

SEQ ID NO: 17 - DNA, 23 bp, Artificial Sequence
  Description of Artificial Sequence Synthetic CHI-B R2 primer
tcytcytcytcytcrtayttktc

SEQ ID NO: 18 - DNA, 23 bp, Artificial Sequence
  Description of Artificial Sequence Synthetic CHI-B R3 primer
ttyttraartayttnswytgraa

SEQ ID NO: 19 - DNA, 23 bp, Artificial Sequence
  Description of Artificial Sequence Synthetic GeneRacer 5′ primer
cgactggagcacgaggacactga

SEQ ID NO: 20 - DNA, 20 bp, Artificial Sequence
  Description of Artificial Sequence Synthetic pCHIB-R1 primer
ctcttacagcactctctagc

SEQ ID NO: 21 - DNA, 26 bp, Artificial Sequence
  Description of Artificial Sequence Synthetic GeneRacer 5′ nested primer
ggacactgacatggactgaaggagta

SEQ ID NO: 22 - DNA, 25 bp, Artificial Sequence
  Description of Artificial Sequence Synthetic GeneRacer 3′ primer
gctgtcaacgatacgctacgtaacg

SEQ ID NO: 23 - DNA, 20 bp, Artificial Sequence
  Description of Artificial Sequence Synthetic PtCHIB-R1 primer
cattgagatacactttctcc

SEQ ID NO: 24 - DNA, 23 bp, Artificial Sequence
  Description of Artificial Sequence Synthetic GeneRacer 3′ nested primer
cgctacgtaacggcatgacagtg

SEQ ID NO: 25 - DNA, 633 bp, Petunia sp.
  CHI-B
atgggaaagaacgaagtgatggtggatgaaattccttttccttctcagttcatgatgactactaagcctttaccattgatgggccatggaataactgacattgagatacactttctccaaattaaattcactgccattggagtttacttggatccagaaattgtaactcatttgcagcagtggaagggtaaatcaggagctgagctaattgaaaatgatgagtttttcgaggctattgttaatgctccagttgataaattcttgagagtagttgtgattaaagaaatcaaaggttcacaatatggagtgcagctagagagtgctgtaagagatcgtttagcagaagtcgataaatatgaagaggaagaagaagaagcacttgagaaaattgttgaatttttccagtctaagtatttcaagaaagattctgtcgtaacatattcttttccagctacttccggcaatgttaagatttcgttcgctacagaaggaaaagaagattcagaaattgaagtgcaaaatgcaaatgttgcaggggagatcaagaaatggtacttaggtggaagtaggggattgtctcctacaactatatcttctttggccaacaccctctctgctgaattatctaaatga

SEQ ID NO: 26 - protein, 210 aa, Petunia sp.
MGKNEVMVDEIPFPSQFMMTTKPLPLMGHGITDIEIHFLQIKFTAIGVYLDPEIVTHLQQWKGKSGAELIENDEFFEAIVNAPVDKFLRVVVIKEIKGSQYGVQLESAVRDRLAEVDKYEEEEEEALEKIVEFFQSKYFKKDSVVTYSFPATSGNVKISFATEGKEDSEIEVQNANVAGEIKKWYLGGSRGLSPTTISSLANTLSAELSK

SEQ ID NO: 27 - DNA, 22 bp, Artificial Sequence
  Description of Artificial Sequence Synthetic PtCHIB-FW primer
atgggaaagaacgaagtgatgg

SEQ ID NO: 28 - DNA, 22 bp, Artificial Sequence
  Description of Artificial Sequence Synthetic PtCHIB-RV primer
tcatttagataattcagcagag

SEQ ID NO: 29 - DNA, 645 bp, Torenia sp.
  TrCHI-B2
atggccggcggtgaagtcgagactgtgatggtggatgagatctcttttcctcgacaaattcaaattactaggcctttgtctttgcttggccatggaatcacagacattgagatacatttcctccaaatcaagttcactgcaatagggatttacttggatctccaaatccttgaccacttacaaaaatggaagggcaaatcagaaaccgagttagcacaagatgatgatttctttgaagccatcgtttcagccccagtggaaaagttcattagagtggtggtgataaaagagatcaagggttcacaatacggagttcagctggagagtgctgttagggaccgtttggctgaggaagacaagtacgaggaggaagaagaatcatctttggaaaaggttgttgatttctttcagtccaaatacttcaggaaagactccgttgtgactcttcacttcccagcttcttcatccattgccgagattgtgtttgcatcaagtgatggtgataaagaagaatcaagaattgaagtgaagaatgcgaacgttgtggagatgattcagaagtggtacttgggtgggacccgggctgtgtctccaaccaccgttgcctctttggccagtggtctttctgctgagttatcgaaatga

SEQ ID NO: 30 - protein, 214 aa, Torenia sp.
MAGGEVETVMVDEISFPRQIQITRPLSLLGHGITDIEIHFLQIKFTAIGIYLDLQILDHLQKWKGKSETELAQDDDFFEAIVSAPVEKFIRVVVIKEIKGSQYGVQLESAVRDRLAEEDKYEEEEESSLEKVVDFFQSKYFRKDSVVTLHFPASSSIAEIVFASSDGDKEESRIEVKNANVVEMIQKWYLGGTRAVSPTTVASLASGLSAELSK

SEQ ID NO: 31 - DNA, 645 bp, Torenia sp.
  TrCHI-B11
atggccggcggtgaagttgagactgtgacggtggatgagatctcttttcctcgacaagttcaaattactaagcctctgtctttggttggccatggaatcacagacattgagatacatttcctccaaatcaagttcactgcaataggaatttacttggatctccaaatcgttgaccacttacaaaaatggaagggcaaatccgaaaccgagttagcaaaagatgacgatttctttgaagccatcgtttcagccccagtggaaaagttctttagggtggtggtgataaaagagatcaagggttcacaatacggagttcagctggagagtgctgttagggaccgtttggctgaggaagacaagtacgaggaggaagaagaagaatctttggaaaaggttgttgaattctttcagtccaaatacttcaggaaagactccgttgtgattcttcacttcccagcttcttcatccattgccgagattgtgttcgcatcgagtgacggtgacagagaagaatcaagaattgaagtgaagaatgcgaacgttgtggagatgatccagaagtggtacttggatgggacccggtctgtatctccaaccaccgtcgcctctttggccagtggtctttatgctgagttatcgaaatga

SEQ ID NO: 32 - protein, 214 aa, Torenia sp.
MAGGEVETVTVDEISFPRQVQITKPLSLVGHGITDIEIHFLQIKFTAIGIYLDLQIVDHLQKWKGKSETELAKDDDFFEAIVSAPVEKFFRVVVIKEIKGSQYGVQLESAVRDRLAEEDKYEEEEEESLEKVVEFFQSKYFRKDSVVILHFPASSSIAEIVFASSDGDREESRIEVKNANVVEMIQKWYLDGTRSVSPTTVASLASGLYAELSK

SEQ ID NO: 33 - DNA, 18 bp, Artificial Sequence
  Description of Artificial Sequence Synthetic TrCHIB-FW primer
gtatggccggcggtgaag

SEQ ID NO: 34 - DNA, 19 bp, Artificial Sequence
  Description of Artificial Sequence Synthetic TrCHIB-RV primer
ctcatttcgataactcagc